(19) United States
(12) Patent Application Publication
(10) Pub. No.: US 2022/0261940 A1
(43) Pub. Date: Aug. 18, 2022

(54) REGISTERED MAIL METHOD AND SYSTEM

(71) Applicant: MOATAZ ABO MOCH, BAGA ALGARBIA (IL)

(72) Inventor: MOATAZ ABO MOCH, BAGA ALGARBIA (IL)

(21) Appl. No.: 17/411,078

(22) Filed: Aug. 25, 2021

(30) Foreign Application Priority Data
Feb. 15, 2021 (IL) 280893

(51) Int. Cl. G06Q 50/26; H04L 12/58; G06Q 10/08; H04L 29/06

(52) U.S. Cl. CPC G06Q 50/265; H04L 51/08; G06Q 20/18; H04L 63/08; G06Q 10/0838

(57) ABSTRACT

A method for sending a registered mail, by a sender, to an addressee, via a registered mail system, the method including the steps of uploading to a cloud storage file(s) to be sent to the addressee, providing to the system a delivery address of the addressee, defining by the sender identification details of the addressee, sending by the system, to the addressee, a hyperlink, upon clicking by the addressee on the hyperlink, identifying the addressee according the identification details, upon positively identifying the addressee, making the file(s) available to the addressee, and sending to the sender a certification of safe delivery of the file(s).

### TECHNICAL FIELD

The invention relates to the field of mail services.

### BACKGROUND

“Registered mail is a mail service offered by postal services in many countries, which allows the sender proof of mailing via a mailing receipt and, upon request, electronic verification that an article was delivered or that a delivery attempt was made.” (From Wikipedia)

Despite the large amount of documents sent by registered mail, the sending and receiving procedure is inefficient, cumbersome and inconvenient for both, sender and recipient. It takes some days until a registered mail is received to its destination locality. Then a letter is sent to the recipient to collect the mail from the post office. Alternatively, the mail is delivered to the addressee's address by a messenger. The whole process may take even an entire week. It requires man work, and accordingly it is money consuming. Not only individuals use registered mail services, but also commercial enterprises, state agencies, etc.

As such, there is a long-felt need for a registered mail system, which simplifies the sending and receiving of registered mail messages.

### SUMMARY

The invention, in embodiments thereof, is directed to a method for sending a registered mail, by a sender, to an addressee, via a registered mail system, the method including the steps of uploading to a cloud storage file(s) to be sent to the addressee, providing to the system a delivery address of the addressee, defining by the sender identification details of the addressee, sending by the system, to the addressee, a hyperlink, upon clicking by the addressee on the hyperlink, identifying the addressee according the identification details, upon positively identifying the addressee, making the file(s) available to the addressee, and sending to the sender a certification of safe delivery of the file(s).

According to one embodiment of the invention, the file(s) are a digital form scanned paper document(s).

The delivery address may be a telephone number, an email address, and a geographical address. When the delivery address is a smartphone, a hyperlink is sent in a message to the smartphone.

The identification details of the addressee may be an ID number, a social security number, a password, a biometric sample, etc.

In one embodiment, the hyperlink points on a computer code adapted to identify the addressee, according to the identification details, before making the file(s) available to the addressee.

In another aspect, the invention is directed to a registered mail system, including a sender module, being a software application executed on a sender's computer and adapted to (a) upload files of a mail item to a cloud storage, and (b) send to an addressee thereof a hyperlink, a receiver module, being a software application executed on an addressee's computer and adapted to (a) execute a code for identifying the addressee upon clicking the hyperlink and make the files of the mail item available to the addressee, and (b) print the files, and a post office module, being a software application executed on a computer operating in a post office, the post office module being adapted to (a) execute a computer code for identifying the addressee, and (b) upon positive identification of the addressee, make the files of the mail item available to the addressee, and print the files.

The computer may be a desktop computer, a smartphone, and the like.

In one embodiment, the sender's computer includes a scanner, for providing a digital form of paper documents to be sent via the registered mail system.

In one embodiment, the post office module is adapted to perform operations of the sender module, for allowing a sender to use the computer operating in a post office for sending registered mail items.

In one embodiment, the computer operating in a post office (“post terminal”) includes a box, a screen installed on the box, a scanner installed on the box, and a printer installed on the box, thereby allowing a user to send and receive registered mail items via the computer in the post office.

In one embodiment, plurality of post terminals is installed in a post office.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF THE DRAWINGS

Embodiments, features, and aspects of the invention are described herein in conjunction with the following drawings in which:

FIG. 1 is a flowchart of a process of sending a mail item to an addressee via a registered mail system, according to the prior art;

FIG. 2 is a block diagram of the major modules of a registered mail system, according to one embodiment of the invention;

FIG. 3 pictorially illustrates a mail terminal of a registered mail system, according to one embodiment of the invention;

FIG. 4 is a flowchart of a process registering a user to an RMS, according to one embodiment of the invention; and

FIG. 5 is a flowchart of a process of sending and receiving a mail item via an RMS, according to one embodiment of the invention.

It should be understood that the drawings are not necessarily drawn to scale.

### DETAILED DESCRIPTION

The invention will be understood from the following detailed description of embodiments which are meant to be descriptive and not limiting. For the sake of brevity, some well-known features, methods, systems, procedures, components, circuits, and so on, are not described in detail.

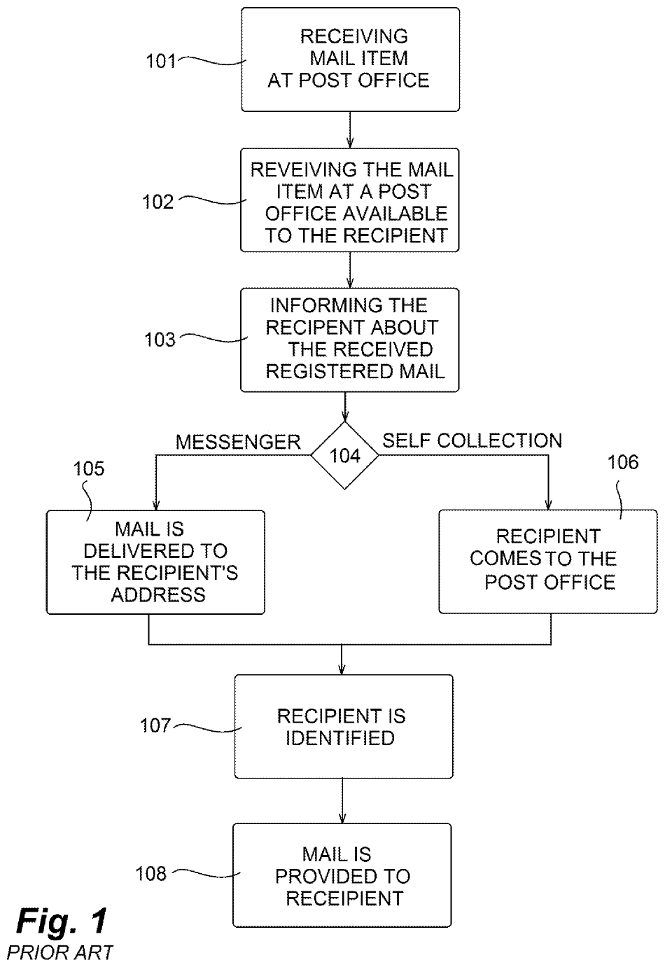

FIG. 1 is a flowchart of a process of sending a mail item to an addressee via a registered mail system, according to the prior art.

At block 101, the sender reaches to a post office (“the sending post office”), provides his mail to the post clerk. The addressee details are registered in a registered mail system, and the mail item is marked by a unique identification number. Then and the mail item is sent to the addressee. Nowadays registered mail system allows the sender to follow the course of the mail item until it is delivered to the addressee.

At block 102, the registered mail item is received at a post office, in which it is available to the addressee (“receiving post office”), for example, at a post office at his neighborhood.

At block 103, the receiving post office informs the addressee about the registered mail waiting for him in the post office.

From block 104, if the addressee prefers to receive the mail at his address, flow continues with block 105; otherwise, i.e., the addressee prefers to collect the registered mail by himself, flow continues with block 106.

At block 107, the addressee meets the post official, which identifies the addressee. The identification is usually carried out according to the identification card of the addressee.

At block 108, the mail is provided to the post official. In addition, the addressee is asked to confirm by his signature that he has received the mail.

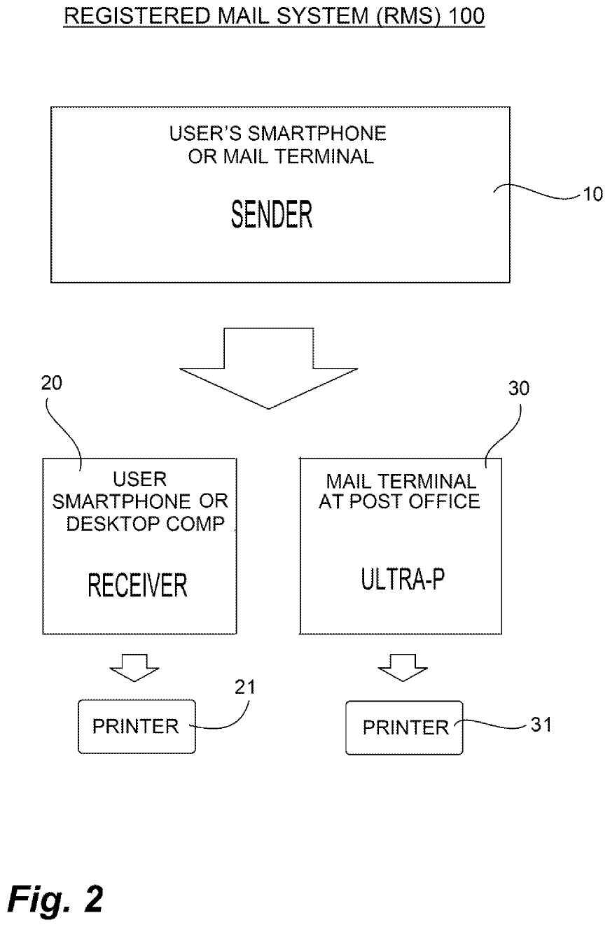

FIG. 2 is a block diagram of the major modules of a registered mail system, according to one embodiment of the invention.

The Registered Mail System (RMS) is marked herein by reference numeral 100.

While in the prior art a mail item is in a form of one or more physical paper sheets, according to an embodiment of the invention the mail item is in a form of one or more digital files. Paper sheets are scanned and stored as digital files.

The first module is referred herein as SENDER and is marked by reference numeral 10. It is an application program executed on a computer such as a smartphone, desktop computer, a web site, etc. Using the Sender module a user sends a registered mail item to an addressee. If the document to be sent is a paper document, the user can scan it, and send the scanned file(s).

The second module is referred herein as RECEIVER and marked by reference numeral 20. It is also an application program executed on a computer such as a smartphone, desktop computer, a web site, etc. Using the Receiver module a user receives registered mail items sent to him by other users using the system, and print it on a printer 21, save them, etc. The Receiver module includes or is connected to a printer 21, on which a received mail item can be printed.

The third module is referred herein as ULURA-P and is marked herein by reference numeral 30. It is an application program executed on a desktop computer operating in a post office. The ULTRA-P module includes or is connected to a printer 31, on which a received mail item can be printed.

Thus, users that have their own smartphone or desktop computer may receive mail messages and print them by their own printer. Other users may print received registered mail items in a post office that uses an ULTRA-P module.

According to a further embodiment of the invention, a single printer serves a plurality of mail terminals.

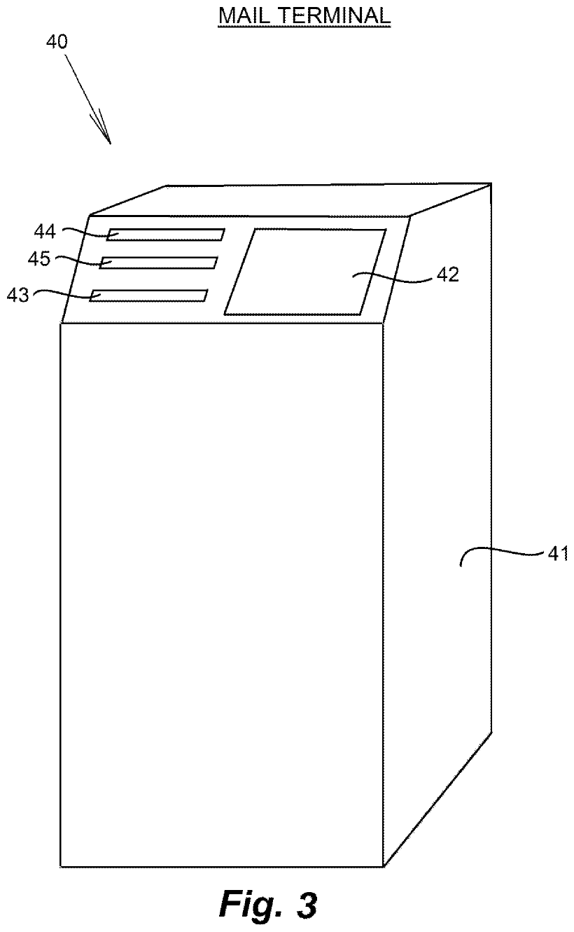

FIG. 3 pictorially illustrates a mail terminal of a registered mail system, according to one embodiment of the invention.

The mail terminal is marked herein by reference numeral 40.

The mail terminal is in a form that reminds an ATM (Automatic Teller Machine). The mail terminal may include a computer that executes an ULTRA-P module (shown in FIG. 2).

The mail terminal includes a box 41, which includes a touch screen 42 for interacting with a user, and a printer. The printer is disposed inside box 41 and therefore is not seen in the figure. The box 41 includes a slot 43 from which printed pages are ejected.

The mail terminal 40 may further include a scanner, by which a user can scan documents to be sent to an addressee using the ULTRA-P system. The figure shows a slot 44 in which a user inserts page(s) to be scanned, and a slot 45 through which scanned pages are ejected.

Thus, the mail terminal can be adapted to be used with the Sender module. As such, a user may send a registered may using the mail terminal by himself, without a help or intervention of post office clerk.

Until now, sending and receiving a registered mail item via a post office required intervention of a post office clerk, time consuming, etc. Using a mail terminal a user can send and receive email items by himself, without an intervention of a post office clerk. Installing several post terminals in a post office shortens the time thereof even more.

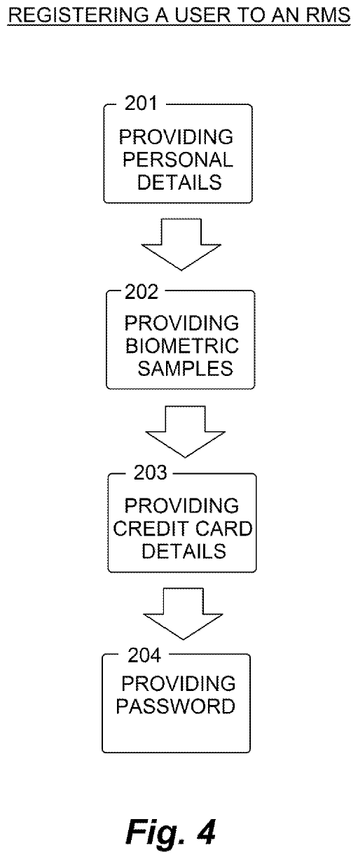

FIG. 4 is a flowchart of a process registering a user to an RMS, according to one embodiment of the invention.

At block 201, a user provides his personal details, such as his name, ID, social security number, address, telephones, etc.

At block 202, the user provides biometric details, for identification. Such biometric details may be, for example, a picture of his face, fingerprint, retina, etc.

At block 203, the user provides his credit card number, or other charging information.

In block 204, the user provides a password.

The password and the biometric samples may be used later for both, entering into the RMS for sending a registered mail, and identification upon receiving a registered mail.

A user can register to an RMS in various ways. If the user uses a computer such as desktop computer and smartphone, he can download an application program through which he can register to the RMS.

Another way to register to an RMS is by browsing a website adapted for this purpose.

A yet another way of registering to an RMS is via a mail terminal, such as the mail terminal illustrated in FIG. 3. The mail terminal is a computer installed in a post office, where it is available to the public.

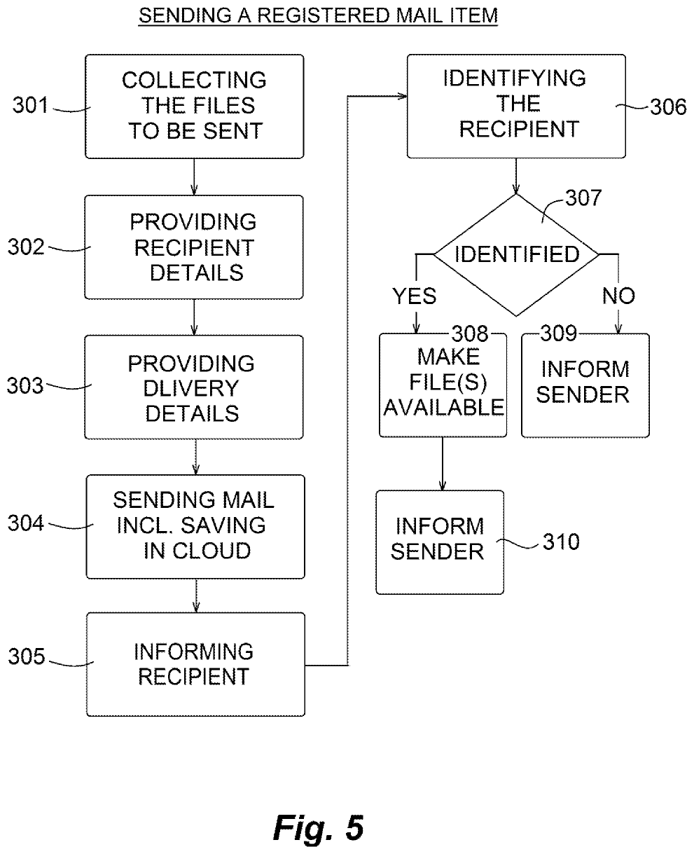

FIG. 5 is a flowchart of a process of sending and receiving a mail item via an RMS, according to one embodiment of the invention.

At block 301, a user collects the files to be sent. If the item to be sent is a paper document, the user scans it into digital files, and puts the digital files in an electronic mail box such as a digital folder.

At block 302, the user provides the details of the addressee, such as telephone number (if he uses a smartphone), as dumb telephone (if he uses only a “dumb” telephone), an email address, or a physical address (if he does not have other receiving means). If the addressee is registered in the RMS, the details of the addressee and the ways to reach him are stored in the RMS's database.

If the recipient is registered to the RMS, the addresses can be identified by his ID or social security number.

At block 303, the sender provides additional delivery details, such as authentication options. An authentication options may be, for example, presenting an identification card or identification number, biometric authentication, his password, etc.

Then, at block 304, the user informs the RMS that the sending process has been ended, by pressing a button for this purpose (“SEND button”).

“Cloud computing is the on-demand availability of computer system resources, especially data storage (cloud storage) and computing power, without direct active management by the user. The term is generally used to describe data centers available to many users over the Internet. (From Wikipedia)

The RMS stores the mail files in “cloud storage”.

In step 305, the registered mail system informs the addressee about the registered mail he has received.

The operation of informing the addressee may be carried out in a variety of ways. If the addressee is registered in the RMS, then his details are available to the RMS, and accordingly can be used to inform the addressee. For example, in the registration process to an RMS a user can specify that he prefers to be informed about new received mail items by SMS, WhatsApp message, email message, a recorded message, etc.

If the addressee does not use any kind of telephone, a letter can be sent to his address, in which he is informed about the new received registered mail.

According to one embodiment of the invention, the message that informs an addressee about a registered mail includes a hyperlink.

In step 306, upon clicking the hyperlink by the recipient, he is asked to provide identification details.

The identification details may be, for example, to provide his ID number, a password, his ID card or ID number, a biometric sample, etc.

From step 307, if the recipient has positively identified as the addressee, flow continues with step 308; otherwise flow continues with step 309, in which the sender is informed that the addressee has not received the registered mail.

In step 308, the received file(s) become available to the recipient, and he can store them, print them, etc.

In step 310, a receipt of delivering the mail is sent to the sender. Preferably, the receipt is digitally signed by the RMS.

It is preferable that the entire process of sending and receiving registered mail via the RMS is encrypted. Using a post terminal may provide to a recipient a higher security level than by using his own computer, since the protection of the computers that run the RMS modules in a post office computer are under the control of the RMS professionals. Thus, if one needs a high level of security in sending a registered mail, he may use a post terminal.

The registered mails may be sent by the RMS in a cloud storage, and be available to both, the recipient and the sender, for further use, such as for proofing that the sent mail was received by the addressee.

API (Application Program Interface) is a set of routines, protocols, and tools for building software applications.

According to an embodiment of the invention, the RMS includes an API, for allowing developing tailored application using the RMS. For example, by using an API, the staff of a HMO (Health Maintenance Organization) can program an application that provides to a doctor an ability to send prescriptions of medicines considered as drugs only to the patient thereof, which upon receiving the registered mail with the prescription is identified.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

1. A method for sending a registered mail, by a sender, to an addressee, via a registered mail system, the method comprising the steps of:
uploading to a cloud storage at least one file to be sent to the addressee;
providing to the system a geographical delivery address of the addressee;
defining by the sender identification details of the addressee;
sending by the system, to the geographical address, via a tangible mail item, a hyperlink;
upon invoking of the hyperlink by the addressee, executing on the cloud a computer code for identifying the addressee according the identification details;
upon positively identifying the addressee, making the at least one file available to the addressee; and
sending to the sender a certification of safe delivery of the at least one file.

2. A method according to claim 1, wherein the at least one file is a digital form of one or more scanned paper documents.

3. (canceled)

4. A method according to claim 1, wherein the identification details of the addressee is selected from a group consisting of: an ID number, a social security number, a password, and a biometric sample.

5. (canceled)

6. A registered mail system, comprising:
a sender module, being a software application executed on a cloud upon being invoked by a sender's computer and adapted to (a) upload files of a mail item to a cloud storage, and (b) send to a geographical address of an addressee thereof a tangible mail item comprising a hyperlink;
a receiver module, being a software application executed on an addressee's computer and adapted to (a) execute on a cloud an identification computer code upon clicking the addressee invoking the hyperlink and make the files of the mail item available to the addressee, and (b) print the files; and
a post office module, being a software application executed on a cloud upon being invoked by a computer operating in a post office, the post office module being adapted to (a) execute computer code for identification of the addressee, and (b) upon positive identification of the addressee, make the files of the mail item available to the addressee, and print the files.

7. A system according to claim 6, wherein the computer is a smartphone.

8. A system according to claim 6, wherein the sender's computer comprises a scanner, for providing a digital form of paper documents to be sent via the registered mail system.

9. A system according to claim 6, wherein the post office module being adapted to perform operations of the sender module, for allowing a sender to use the computer operating in a post office for sending registered mail items.

10. A system according to claim 6, wherein the computer operating in a post office comprising:
a box;
a screen installed on the box;
a scanner installed on the box; and
a printer installed on the box,
thereby allowing a user to send and receive registered mail items via the computer.

11. A system according to claim 10, wherein the post office further comprising at least one additional computer operating in the post office, and comprising:
a box;
a screen installed on the box;
a scanner installed on the box; and
a printer installed on the box,
thereby allowing a user to send and receive registered mail items via the terminal.

12. A method according to claim 4, wherein said biometric sample is selected from a group consisting of: a picture of a face, a picture of a fingerprint, and a picture of a retina.
